(19) United States
(12) Patent Application Publication
(10) Pub. No.: US 2015/0353564 A1
(43) Pub. Date: Dec. 10, 2015

(54) Processes for the Preparation of Chiral Beta Amino Acid Derivatives Using Asymmetric Hydrogenation Catalysts

(71) Applicant: APOTEX TECHNOLOGIES INC., Toronto (CA)

(72) Inventors: Kamaluddin Abdur-Rashid, Mississauga (CA); Rongwei Guo, Oakville (CA); Xuanhua Chen, Oakville (CA); Stephen E. Horne, Burlington (CA)

(21) Appl. No.: 14/762,280

(22) PCT Filed: Jan. 14, 2014
(86) PCT No.: PCT/CA2014/000013

§ 371 Date: Jul. 21, 2015

Related U.S. Application Data
(60) Provisional application No. 61/755,290, filed on Jan. 22, 2013.

(51) Int. Cl. C07D 487/04

(52) U.S. Cl. CPC C07D 487/04

(57) ABSTRACT

This invention provides processes for the preparation of Sitagliptin and pharmaceutically acceptable salts thereof, said processes including enantioselective hydrogenation of a prochiral enamine using chiral ruthenium catalyst.

### TECHNICAL FIELD

The present invention relates to processes for the preparation of Sitagliptin, which is useful as a medicament.

### BACKGROUND

Sitagliptin (I) is marketed in the United States as its phosphate salt under the trade name Januvia™, and is indicated for the treatment of diabetes mellitus type 2.

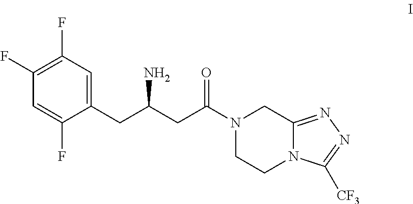 [SMILES: N[C@@H](CC(=O)N1CCn2c(nnc2C(F)(F)F)C1)Cc1cc(F)c(F)cc1F]

WO 2011113399 discloses a method of preparing Sitagliptin, comprising hydrogenation of an enamino-amide precursor, wherein the hydrogenation is carried out in a suspension or a solution and is catalyzed by a complex compound formed of Ru and an (R) or (S)-pseudo-o-bisphosphino-[2,2]-paracyclophane ligand (Scheme 1).

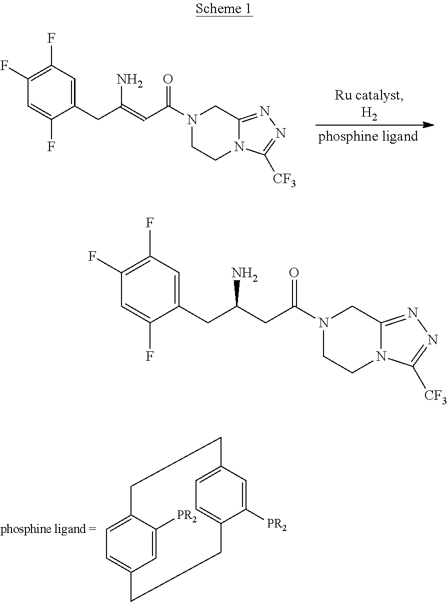

Steinhuebel et al. disclose in J. Am. Chem. Soc., 2009, 131, 32, 11316-11317 that asymmetric reductive amination of β-keto amides catalyzed by the chiral catalyst Ru(OAc)₂((R)-dm-segphos) produces unprotected β-amino amides with high yields and high enantioselectivities (94.7-99.5% ee). This “one-pot” methodology has been successfully employed to produce Sitagliptin with 99.5% ee and 91% assay yield (Scheme 2).

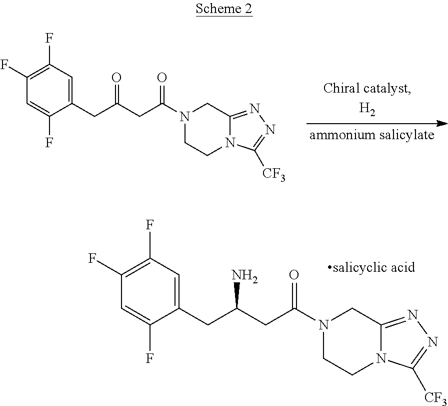 [SMILES: O=C(CC(=O)N1CCn2c(nnc2C(F)(F)F)C1)Cc1cc(F)c(F)cc1F ; N[C@@H](CC(=O)N1CCn2c(nnc2C(F)(F)F)C1)Cc1cc(F)c(F)cc1F]

Hansen et al. report in J. Am. Chem. Soc., 2009, 131, 25, 8798-8804 that a highly efficient synthesis of Sitagliptin has been developed. The key dehydroSitagliptin intermediate is prepared in three steps in one pot and directly isolated in 82% yield and >99.6 wt % purity. Highly enantioselective hydrogenation of dehydroSitagliptin, with as low as 0.15 mol % of Rh(I)/Bu JOSIPHOS, affords Sitagliptin (Scheme 3).

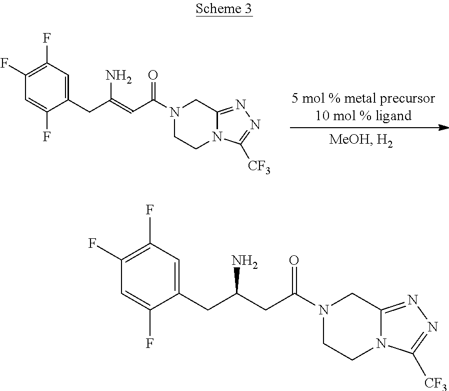 [SMILES: N/C(=C\C(=O)N1CCn2c(nnc2C(F)(F)F)C1)Cc1cc(F)c(F)cc1F ; N[C@@H](CC(=O)N1CCn2c(nnc2C(F)(F)F)C1)Cc1cc(F)c(F)cc1F]

WO 2006081151 discloses a process for the efficient preparation of enantiomerically enriched beta amino acid derivatives which are useful in the asymmetric synthesis of biologically active molecules. The process comprises an enantioselective hydrogenation of a prochiral beta amino acrylic acid derivative substrate in the presence of an ammonium salt and a transition metal precursor complexed with a chiral ferrocenyl diphosphine ligand.

WO 2005097733 discloses a process for the efficient preparation of enantiomerically enriched beta amino acid derivatives wherein the amino group is unprotected. The product chiral beta amino acid derivatives are useful in the asymmetric synthesis of biologically active molecules. The process comprises an enantioselective hydrogenation of an amine-unprotected prochiral beta-amino acrylic acid or derivative thereof in the presence of a rhodium metal precursor complexed with a chiral mono- or bisphosphine ligand.

U.S. Pat. No. 7,468,459 discloses a process for the efficient preparation of enantiomerically enriched beta amino acid derivatives which are useful in the asymmetric synthesis of biologically active molecules. The process disclosed therein comprises an enantioselective hydrogenation of a prochiral beta amino acrylic acid derivative substrate in the presence of a transition metal precursor complexed with a chiral ferrocenyl diphosphine ligand.

### SUMMARY

The present invention provides processes for the enantioselective hydrogenation of a prochiral enamine of the formula II, providing Sitagliptin of formula I. In an illustrative embodiment of the present invention, Sitagliptin may be prepared by an exemplary process as set out in Scheme 5. Exemplary reagents and conditions for these reactions are disclosed herein.

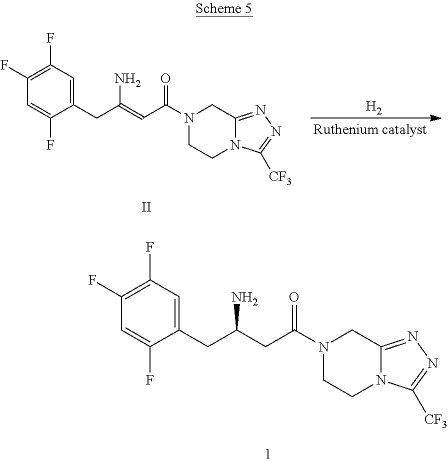 [SMILES: N/C(=C\C(=O)N1CCn2c(nnc2C(F)(F)F)C1)Cc1cc(F)c(F)cc1F ; "H2" ; "II" ; N[C@@H](CC(=O)N1CCn2c(nnc2C(F)(F)F)C1)Cc1cc(F)c(F)cc1F]

The present invention provides a facile preparation of Sitagliptin, in high enantiomeric excess using conditions including hydrogen gas pressures that are suitable for industrial manufacturing scale.

According to illustrative embodiments of the present invention, there is provided a process for the enantioselective preparation of Sitagliptin of Formula I or a pharmaceutically acceptable salt thereof comprising hydrogenation of a compound of the Formula II:

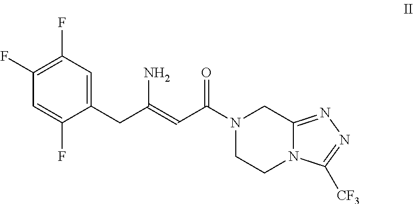 [SMILES: N/C(=C\C(=O)N1CCn2c(nnc2C(F)(F)F)C1)Cc1cc(F)c(F)cc1F]

in the presence of a ruthenium catalyst, wherein the catalyst is selected from the group consisting of:

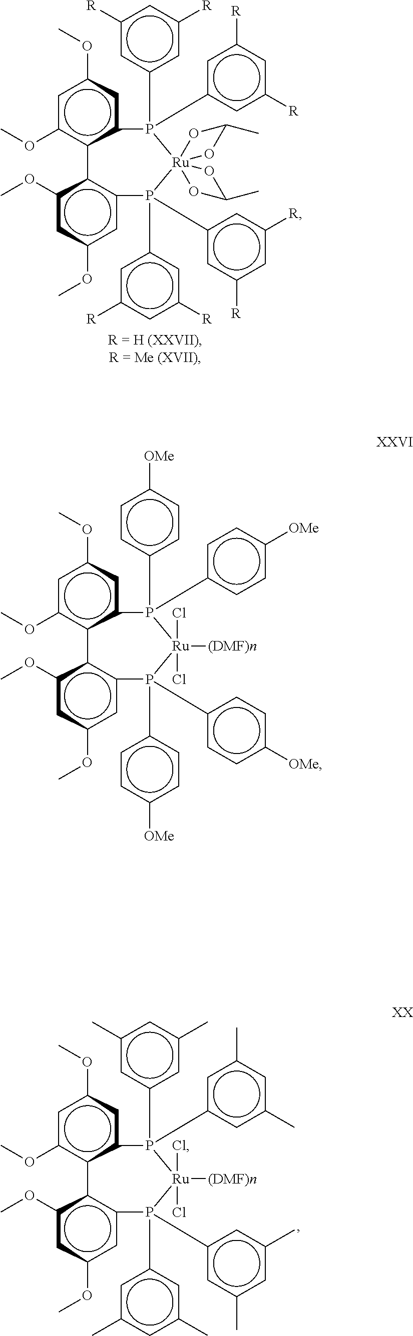

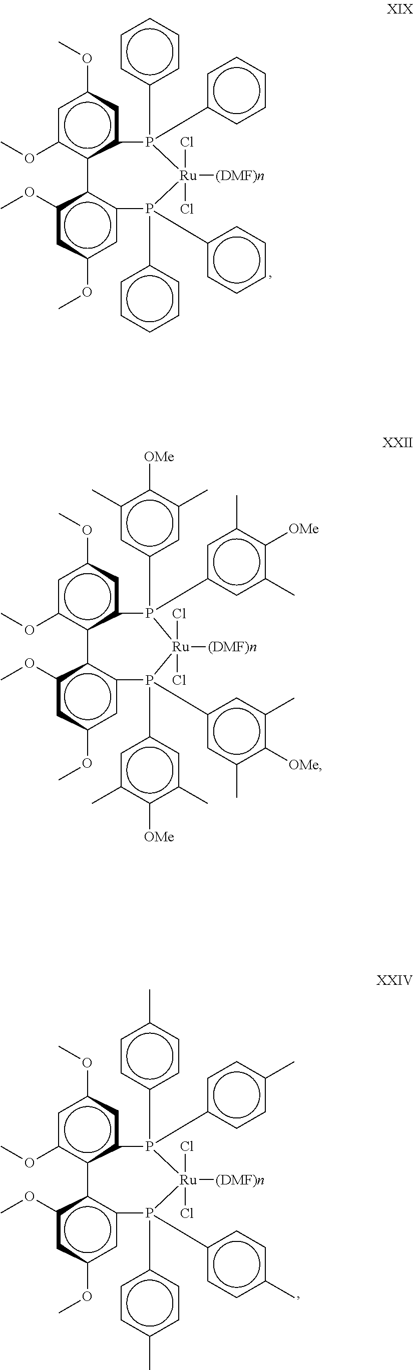

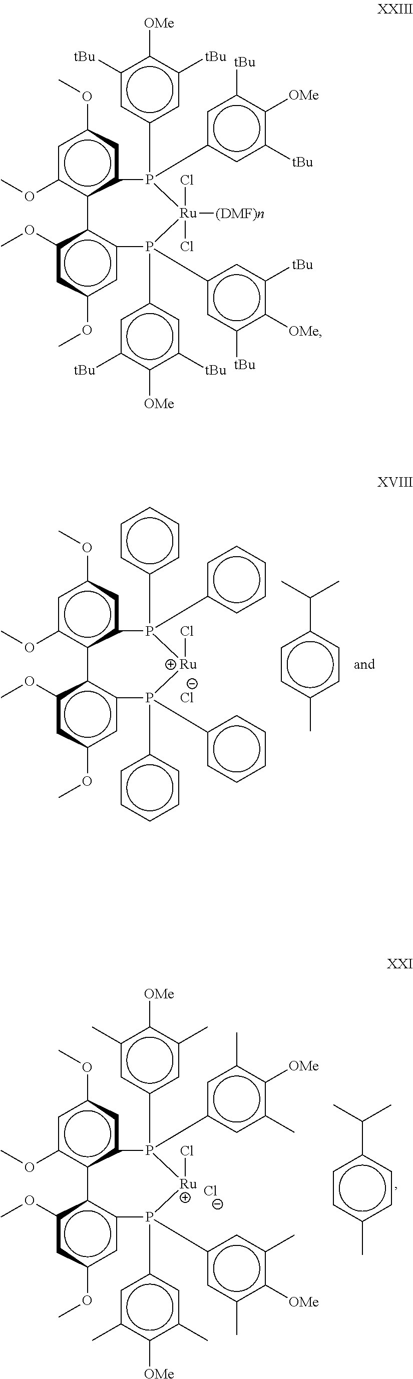

wherein n is 1 or 2.

Other aspects and features of the present invention will become apparent to those ordinarily skilled in the art upon review of the following description of specific embodiments of the invention in conjunction with the accompanying figures.

### DETAILED DESCRIPTION

As used herein, the term enantioselective when used in reference to a reaction, means a reaction in which the preferred enantiomer of a chiral product is produced in an enantiomeric excess of at least about 70% with respect to the non-preferred enantiomer.

According to illustrative embodiments of the present invention, there is provided a process for the enantioselective preparation of Sitagliptin of Formula I or a pharmaceutically acceptable salt thereof comprising hydrogenation of a compound of the Formula II:

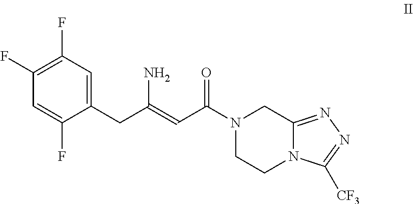 [SMILES: N/C(=C\C(=O)N1CCn2c(nnc2C(F)(F)F)C1)Cc1cc(F)c(F)cc1F]

in the presence of a ruthenium catalyst, wherein the catalyst is selected from the group consisting of:

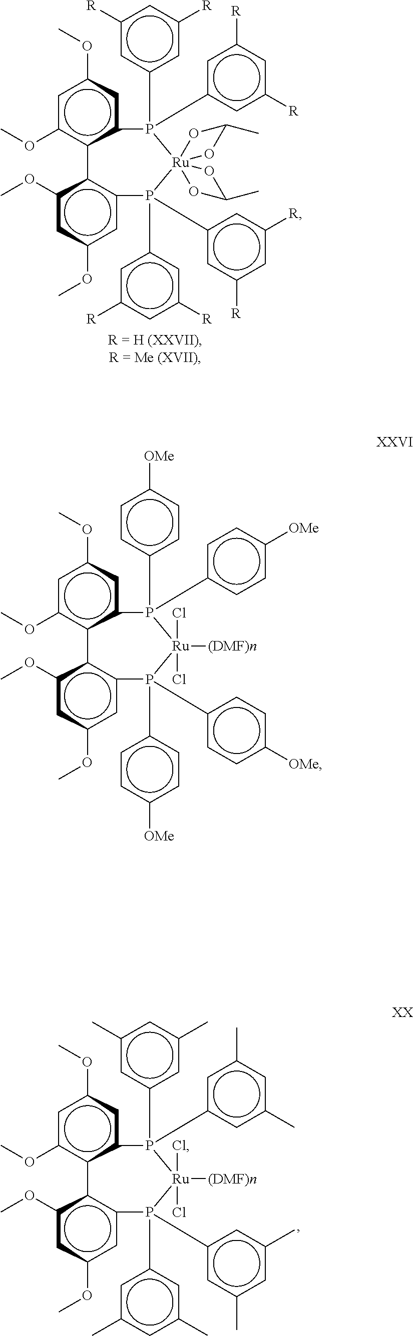

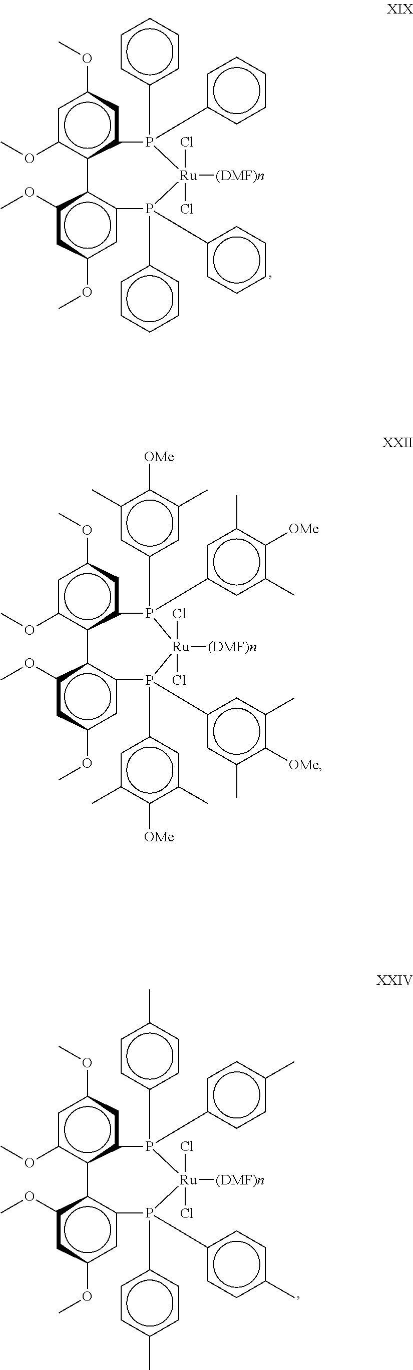

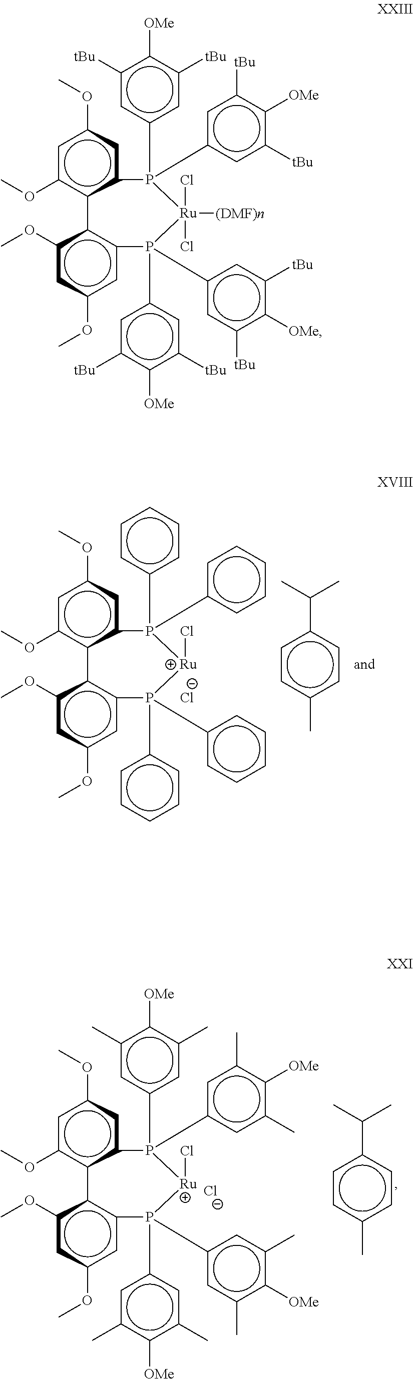

wherein n is 1 or 2.

The hydrogenation of the compound of Formula II may be conducted in the presence of an acid wherein the acid may be an organic acid or an inorganic acid. The organic acid may be selected from the group consisting of acetic acid, chloroacetic acid, and salicylic acid. The amount of acid may be from about 0.5 molar equivalents to about 3 molar equivalents with respect to the compound of Formula II.

The hydrogenation of the compound of Formula II may be conducted in the presence of an ammonium salt. The ammonium salt may be selected from the group consisting of ammonium acetate, ammonium dihydrogen phosphate, and ammonium salicylate. The amount of ammonium salt may be from about 0.1 molar equivalents to about 5 molar equivalents with respect to the compound of Formula II.

The substrate to catalyst molar ratio may be from about 1000:1 to about 1:1. In some embodiments, the substrate to catalyst molar ratio may be from about 200:1 to about 100:1.

The suitable hydrogenation catalyst may be finely dispersed solids or adsorbed on an inert support such as carbon or alumina. The hydrogenation may be performed by using hydrogen gas or transfer hydrogenation. It should also be noted that catalyst moistened with water, for instance 50% water wet ruthenium catalyst, is also suitable.

The hydrogenation of the compound of Formula II may be conducted in a suitable solvent. The suitable solvent may be a protic or an aprotic organic solvent. The suitable solvent may be selected from the group consisting of alcohols (e.g. methanol, ethanol, propanol, isopropanol, butanol), alkyl ethers (e.g. tetrahydrofuran, dioxane, diethyl ether, methyl ethyl ether, methyl t-butyl ether, diisopropyl ether, butyl ether), alkyl esters (e.g. ethyl acetate, isopropyl acetate), aromatic hydrocarbons (e.g. benzene, toluene, xylenes, hexanes and heptanes), and mixtures thereof. In some embodiments, the suitable solvent may be selected from the group consisting of methanol, ethanol, 2-propanol, toluene, tetrahydrofuran, and ethyl acetate.

The hydrogenation of the compound of Formula II may be conducted under an absolute hydrogen pressure ranging from about 10 psi to about 250 psi. In many embodiments, the absolute pressure may be from about 90 psi to about 120 psi.

The hydrogenation of the compound of Formula II may be conducted at a temperature ranging from about 20° C. to about 150° C., over a period ranging from about 1 hour to about 72 hours. In many embodiments, the temperature ranges from about 80° C. to about 100° C.

The compound of the formula I obtained by the hydrogenation may have a chiral purity (enantiomeric excess, or e.e.%) greater than 70%, greater than 95% and greater than 99%.

The compound of formula I can be isolated from the reaction mixture as its free base form, or as a pharmaceutically acceptable salt.

### Examples

The following examples are illustrative of some of the embodiments of the invention described herein. These examples should not be considered to limit the spirit or scope of the invention in any way.

### General Experimental Conditions

All preparations and manipulations of catalysts were carried out under hydrogen or argon atmospheres with the use of standard Schlenk, vacuum line and glove box techniques in dry, oxygen-free solvents. Tetrahydrofuran (THF), toluene, dichloromethane, diethyl ether (Et₂O) and hexanes were purified and dried using an Innovative Technologies solvent purification system. Methanol, ethanol and 2-propanol were dried by refluxing and distilling over the respective magnesium alkoxide, and collecting and storing the solvent over activated molecular sieves. Chiral diphosphines were obtained from Kanata Chemical Technologies Inc. Deuterated solvents were degassed and dried before use. NMR spectra were recorded on a Varian Unity Inova 300 MHz spectrometer (300 MHz for ¹H, 75 MHz for ¹³C and 121.5 for ³¹P). All ³¹P chemical shifts were measured relative to 85% H₃PO₄ as an external reference. The ¹H and ¹³C chemical shifts were measured relative to partially deuterated solvent peaks but are reported relative to tetramethylsilane. Hydrogenation reactions were performed using a Parr Series 5000 Multi Reactor system with 50 mL pressure reactors, or a 600 mL Parr Pressure Reactor. An Agilent Technologies Series 1200 HPLC system was used to analyze the reaction mixtures, standards and isolated products.

### Analysis

Preparation of Sitagliptin standard: Sitagliptin free base (40 mg), salicylic acid (1 equiv) and ammonium salicylate (3 equiv.) were dissolved in methanol (25 mL). The concentration of the standard sample was 1.6 mg/mL.

Preparation of enamine amide standard: The substrate (40 mg) was dissolved in methanol (25 mL). The concentration of the standard sample was 1.6 mg/mL.

HPLC conditions: All analyses were performed on an Agilent 1200 HPLC using the following conditions: Column: Diacel Chiralpak AD-H column (250 mm×4.6 mm, particle size: 5 μm)

Eluent: Methanol/Hexane/Dimethylamine=600/400/1;

Flow rate: 0.8 mL/min;

Injection volume: 5 μL;

Column temperature: 35° C.;

UV detection: 268 nm;

Retention Times: 8.72 min. (enamine); 12.4 min. (S)-isomer; 14.0 min. (R)-isomer

Calculation of conversion: Conv %=(A_{ssub}−A_{lsub})/Assub−100. (A_{ssub}=peak area of standard substrate; A_{lsub}=peak area of substrate remaining in the reaction mixture).

Calculation of yield: Yield %=Aₚ/Aₛ×100. (Aₚ=peak area of product; Aₛ=peak area of Sitagliptin standard).

### Example 1a

The enamine substrate of formula II (400 mg, 1.0 mmol), methanol (2.0 mL) and acetic acid (60 mg, 1.0 mmol) were added to a 50 mL Parr Multireactor autoclave. The mixture was degassed 10 times with argon. Ruthenium catalyst XVIII (1.0×10⁻² mmol) was dissolved in methanol (2.0 mL) and injected under a stream of argon gas into the autoclave. The resulting reaction mixture was degassed 5 times with hydrogen. The temperature of the autoclave was set to 80° C. and the hydrogen pressure was set to 235 psi. The reaction mixture was stirred for 17 hours. A sample of the reaction mixture was withdrawn from the autoclave, dissolved in methanol and filtered through silica to remove the catalyst. The conversion and chiral selectivity were determined using the established analytical method. Conversion=>99%; enantiomeric excess=98.2%.

### Example 1b

The procedure described in Example 1a was followed, but employing catalyst XVII. Conversion=>99%; enantiomeric excess=94.7%.

### Example 1c

The procedure described in Example 1a was followed, but employing catalyst XIX. Conversion=>99%; enantiomeric excess=98.0%.

### Example 1d

The procedure described in Example 1a was followed, but employing catalyst XX. Conversion=>99%; enantiomeric excess=95.2%.

### Example 1e

The procedure described in Example 1a was followed, but employing catalyst XXI. Conversion=>99%; enantiomeric excess=95.7%.

### Example 1f

The procedure described in Example 1a was followed, but employing catalyst XXII. Conversion=>99%; enantiomeric excess=97.0%.

### Example 1g

The procedure described in Example 1a was followed, but employing catalyst XXIII. Conversion=>99%; enantiomeric excess=92.0%.

### Example 1h

The procedure described in Example 1a was followed, but employing catalyst XXIV. Conversion=>99%; enantiomeric excess=98.0%.

### Example 1i

The procedure described in Example 1a was followed, but employing catalyst XXVI. Conversion=>99%; enantiomeric excess=98.2%.

### Example 2a

The enamine substrate (400 mg, 1.0 mmol), methanol (2.0 mL), salicylic acid (1 mmol, 1 equivalent), and ammonium salicylate (3 mmol, 3 equivalents) were added to a 50 mL Parr Multireactor autoclave. The mixture was degassed 10 times with argon. Ruthenium catalyst XIX (1.0×10⁻² mmol) was dissolved in methanol (2.0 mL) and injected under a stream of argon gas into the autoclave. The resulting reaction mixture was degassed 5 times with hydrogen. The temperature of the autoclave was set to 80° C. and the hydrogen pressure was set to 235 psi. The reaction mixture was stirred for 20 hours. The contents of the autoclave (from 400 mg substrate) were cooled and diluted with methanol to 50 mL. A 1.0 mL aliquot was then diluted to 5.0 mL with methanol. Conversion of II was assayed by HPLC by comparing the peak area of remaining substrate with that of a standard sample. Yield of I was assayed by HPLC by comparing the peak area of product with that of a standard sample. Enantiomeric excess was assayed by chiral HPLC. Conversion=100%; yield=76.2%; enantiomeric excess=98.9%.

### Example 2b

The procedure described for Example 2a was followed, but using no additives. Conversion=78.0%; yield=25.0%; enantiomeric excess=96.7%.

### Example 2c

The procedure described for Example 2a was followed, but using chloroacetic acid (1 equivalent) in place of salicylic acid, and no ammonium salt. Conversion=96.0%; yield=58.3%; enantiomeric excess=98.6%.

### Example 2d

The procedure described for Example 2a was followed, but using no ammonium salt. Conversion=95.5%; yield=59.0%; enantiomeric excess=98.7%.

### Example 2e

The procedure described for Example 2a was followed, but using ammonium acetate (3 equivalents) in place of ammonium salicylate. Conversion=98.5%; yield=41.0%; enantiomeric excess=95.7%.

### Example 2f

The procedure described for Example 2a was followed, but using acetic acid (1 equivalent) in place of salicylic acid, and no ammonium salt. Conversion=100%; yield=47.0%; enantiomeric excess=97.5%.

### Example 2g

The procedure described for Example 2a was followed, but using acetic acid (1 equivalent) in place of salicylic acid, and ammonium acetate (3 equivalents) in place of ammonium salicylate. Conversion=100%; yield=36.5%; enantiomeric excess=97.0%.

### Example 2h

The procedure described for Example 2a was followed, but using no acid additive, and ammonium dihydrogen phosphate (1 equivalent) in place of ammonium salicylate. Conversion=100%; yield=34.5%; enantiomeric excess=96.9%.

### Example 2i

The procedure described for Example 2a was followed, but using ammonium dihydrogen phosphate (1 equivalent) in place of ammonium salicylate. Conversion=100%; yield=62.4%; enantiomeric excess=98.3%.

### Example 2j

The procedure described for Example 2a was followed, but using no acid additive, and a reaction time of 5 hours. Conversion=86.0%; yield=36.0%; enantiomeric excess=98.7%.

### Example 3a

The enamine substrate (400 mg, 1.0 mmol), ethyl acetate (2.0 mL), salicylic acid (1 equivalent), and ammonium salicylate (3 equivalents) were added to a 50 mL Parr Multireactor autoclave. The mixture was degassed 10 times with argon. Ruthenium catalyst XXVII (1.0×10⁻² mmol) was dissolved in ethyl acetate (2.0 mL) and injected under a stream of argon gas into the autoclave. The resulting reaction mixture was degassed 5 times with hydrogen. The temperature of the autoclave was set to 75° C. and the hydrogen pressure was adjusted to 235 psi. The reaction mixture was stirred for 27 hours. The contents of the autoclave (from 400 mg substrate) were cooled and diluted with methanol to 50 mL. A 1.0 mL aliquot was then diluted to 5.0 mL with methanol. Conversion of II was assayed by HPLC by comparing the peak area of remaining substrate with that of a standard sample. Yield of I was assayed by HPLC by comparing the peak area of product with that of a standard sample. Enantiomeric excess was assayed by chiral HPLC. Conversion=100%; yield=83.2%; enantiomeric excess=99.7%.

### Example 3b

The procedure of Example 3a was followed, but using methanol as the solvent and a reaction time of 20 hours. Conversion=100%; yield=85.6%; enantiomeric excess=98.3%.

### Example 3c

The procedure of Example 3a was followed, but using ethanol as the solvent and a reaction time of 20 hours. Conversion=100%; yield=85.8%; enantiomeric excess=99.0%.

### Example 3d

The procedure of Example 3a was followed, but using toluene as the solvent and a reaction time of 20 hours. Conversion=82.0%; yield=62.0%; enantiomeric excess=99.9%.

### Example 3e

The procedure of Example 3a was followed, but using tetrahydrofuran as the solvent and a reaction time of 20 hours. Conversion=80.0%; yield=57.8%; enantiomeric excess=99.9%.

### Example 3f

The procedure of Example 3a was followed, but using 2-propanol as the solvent and a reaction time of 20 hours. Conversion=100%; yield=93.2%; enantiomeric excess=99.3%.

### Example 4a

The enamine substrate (400 mg, 1.0 mmol), 2-propanol (2.0 mL) and a mixture of salicylic acid (1 equivalent)/ammonium salicylate (3 equivalents) (SA/NH₄SA) were added to a 50 mL Parr Multireactor autoclave. The mixture was degassed 10 times with argon. Ruthenium catalyst XXVII (1.0×10⁻² mmol) was dissolved in 2-propanol (2.0 mL) and injected under a stream of argon gas into the autoclave. The resulting reaction mixture was degassed 5 times with hydrogen. The temperature of the autoclave was set to 80° C. and the hydrogen pressure was adjusted to 80 psi. The reaction mixture was stirred for 17 hours. The contents of the autoclave (from 400 mg substrate) were cooled and diluted with methanol to 50 mL. A 1.0 mL aliquot was then diluted to 5.0 mL with methanol. Conversion of II was assayed by HPLC by comparing the peak area of remaining substrate with that of a standard sample. Yield of I was assayed by HPLC by comparing the peak area of product with that of a standard sample. Enantiomeric excess was assayed by chiral HPLC. Conversion=98.5%; yield=88.0%; enantiomeric excess=99.3%.

### Example 4b

The procedure of Example 4a was followed, but using a hydrogen pressure of 250 psi, a substrate to catalyst molar ratio of 500:1, and a reaction time of 63 hours. Conversion=98.5%; yield=73.6%; enantiomeric excess=98.5%.

### Example 4c

The procedure of Example 4a was followed, but using a hydrogen pressure of 50 psi. Conversion=91.6%; yield=80.0%; enantiomeric excess=99.3%.

### Example 4d

The procedure of Example 4a was followed, but using a hydrogen pressure of 100 psi, and a substrate to catalyst molar ratio of 200:1. Conversion=88.4%; yield=73.8%; enantiomeric excess=98.7%.

### Example 5

The enamine substrate (400 mg, 1.0 mmol), a 1:3 mixture of isopropanol:toluene (2.0 mL) and a mixture of salicylic acid (1 equivalent)/ammonium salicylate (4 equivalents) (SA/NH₄SA) were added to a 50 mL Parr Multireactor autoclave. The mixture was degassed 10 times with argon. Ruthenium catalyst XXVII (0.5×10⁻² mmol) was dissolved in a 1:3 mixture of isopropanol:toluene (2.0 mL) and injected under a stream of argon gas into the autoclave. The resulting reaction mixture was degassed 5 times with hydrogen. The temperature of the autoclave was set to 90° C. and the hydrogen pressure was adjusted to 85 psi (working pressure=103 psi). The reaction mixture was stirred for 17 hours. The contents of the autoclave were cooled and diluted with methanol to 50 mL. A 1.0 mL aliquot was then diluted to 5.0 mL with methanol. Conversion of II was assayed by HPLC by comparing the peak area of remaining substrate with that of a standard sample. Yield of I was assayed by HPLC by comparing the peak area of product with that of a standard sample. Enantiomeric excess was assayed by chiral HPLC. Conversion=99.0%; yield=81.2%; enantiomeric excess=99.1%.

### Example 6

The enamine substrate (400 mg, 1.0 mmol), toluene (2.0 mL), salicylic acid (1 mmol), and ammonium salicylate (0.5 mmol) were added to a 50 mL Parr Multireactor autoclave. The mixture was degassed 10 times with argon. Ruthenium catalyst XXVII (0.5×10⁻² mmol) was dissolved in toluene (2.0 mL) and injected under a stream of argon gas into the autoclave. The resulting reaction mixture was degassed 5 times with hydrogen. The temperature of the autoclave was set to 95° C. and the hydrogen pressure was adjusted to 92 psi (working pressure=103 psi). The reaction mixture was stirred for 17 hours. The contents of the autoclave (from 400 mg substrate) were cooled and diluted with methanol to 50 mL. A 1.0 mL aliquot was then diluted to 5.0 mL with methanol. Conversion of II was assayed by HPLC by comparing the peak area of remaining substrate with that of a standard sample. Yield of I was assayed by HPLC by comparing the peak area of product with that of a standard sample. Enantiomeric excess was assayed by chiral HPLC. Conversion=97.0%; yield=89.0%; enantiomeric excess=99.3%.

### Example 7

To a solution of the free amine (5.0 g, 12.3 mmol) in 2-propanol (21 mL) and water (6 mL) was added 45 wt % H₃PO₄ (13.5 mmol) dropwise. The resulting clear solution was seeded (with solid obtained by evaporating a small sample of the reaction mixture to dryness) and stirred at room temperature. The reaction mixture slowly became cloudy, followed by precipitation of a white solid. The suspension was stirred for 30 minutes and another portion of 2-propanol (20 mL) was added. The resulting slurry was warmed to 60° C. and stirred for 1 hour. The reaction mixture was slowly cooled to room temperature and stirred overnight. The suspension was then filtered and washed with aqueous 2-propanol (20 wt. % water, 15 mL) and dried under vacuum to give the product as a white solid. Yield=5.95 g, 92.6%. Chemical purity: >99.9% (HPLC); e.e. >99.9% (R-enantiomer).

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. A process for the enantioselective preparation of Sitagliptin of Formula I or a pharmaceutically acceptable salt thereof comprising hydrogenation of a compound of Formula II: 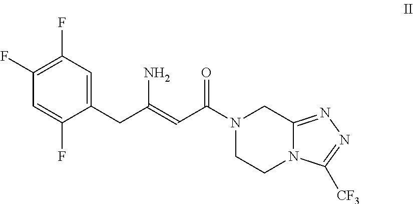 [SMILES: N/C(=C\C(=O)N1CCn2c(nnc2C(F)(F)F)C1)Cc1cc(F)c(F)cc1F]
in the presence of a ruthenium catalyst, wherein the catalyst is selected from the group consisting of: 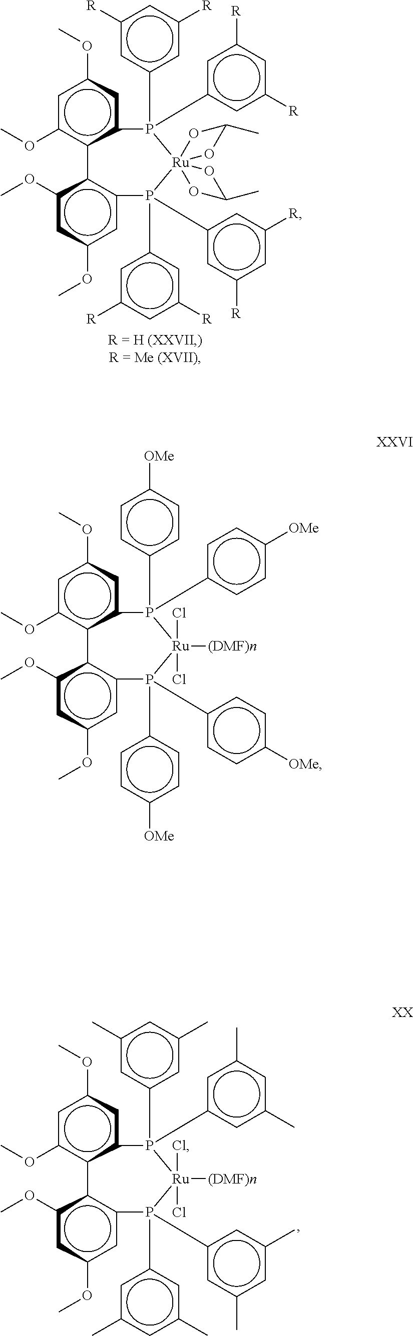 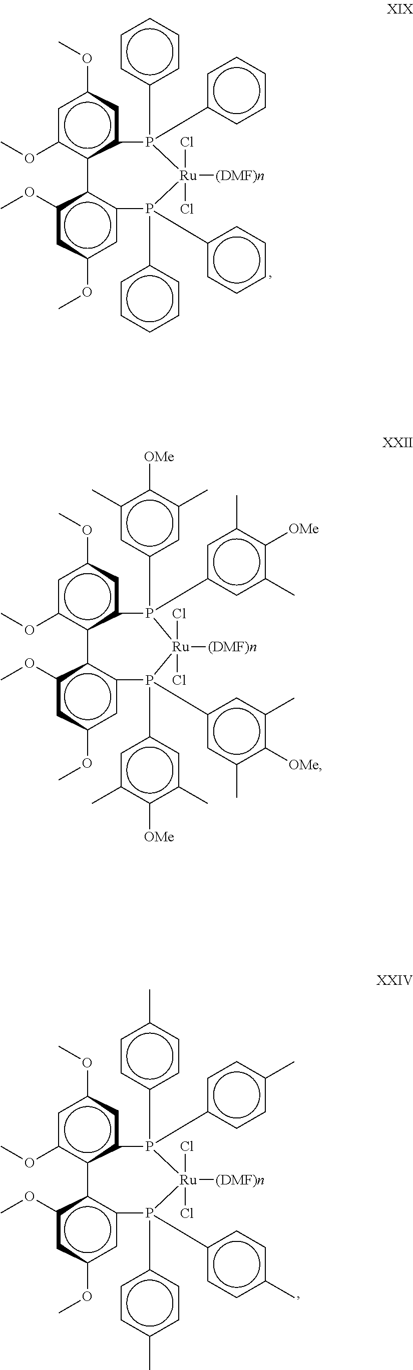 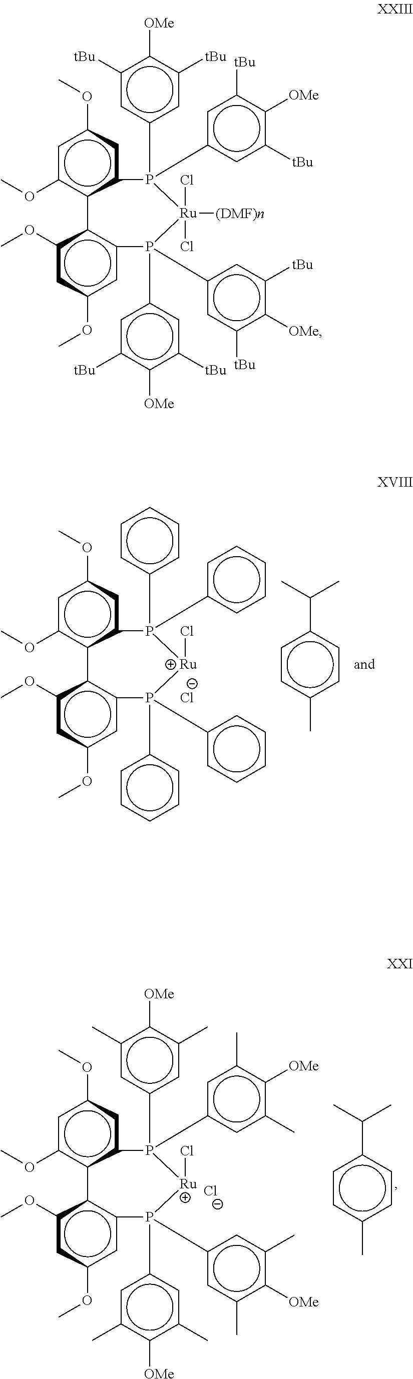
wherein n is 1 or 2.

2. The process of claim 1 wherein the ruthenium catalyst is a catalyst of the Formula XXVII.

3. The process of claim 1 wherein the hydrogenation is conducted in the presence of an acid.

4. The process of claim 3 wherein the acid is selected from the group consisting of acetic acid, chloroacetic acid, and salicylic acid.

5. The process of claim 3 wherein the hydrogenation is conducted in the presence of an ammonium salt.

6. The process of claim 5 wherein the ammonium salt is selected from the group consisting of ammonium acetate, ammonium dihydrogen phosphate, and ammonium salicylate.

7. The process of claim 1 wherein the hydrogenation is conducted in a solvent selected from the group consisting of methanol, ethanol, 2-propanol, toluene, tetrahydrofuran, ethyl acetate, and mixtures thereof.

8. The process of claim 1 wherein Sitagliptin of Formula I is formed in an enantiomeric excess of at least 90% with respect to the (S)-enantiomer of Sitagliptin.

9. The process of claim 1 wherein Sitagliptin of Formula I is formed in an enantiomeric excess of at least 99% with respect to the (S)-enantiomer of Sitagliptin.

10. The process of claim 1 wherein Sitagliptin of Formula I is formed in an enantiomeric excess of at least 99.8% with respect to the (S)-enantiomer of Sitagliptin.

11. The process of claim 1 wherein the substrate to catalyst molar ratio is about 200 to 1.

12. The process of claim 7 wherein the substrate to catalyst molar ratio is about 200 to 1.

13. The process of claim 4 wherein the hydrogenation is conducted in the presence of an ammonium salt.

14. The process of claim 3 wherein the hydrogenation is conducted in a solvent selected from the group consisting of methanol, ethanol, 2-propanol, toluene, tetrahydrofuran, ethyl acetate, and mixtures thereof.

15. The process of claim 6 wherein the hydrogenation is conducted in a solvent selected from the group consisting of methanol, ethanol, 2-propanol, toluene, tetrahydrofuran, ethyl acetate, and mixtures thereof.
